(19) United States
(12) Patent Application Publication
(10) Pub. No.: US 2021/0120870 A1
(43) Pub. Date: Apr. 29, 2021

(54) STERILIZATION BOX AND ELECTRONIC CIGARETTE ASSEMBLY THEREOF

(71) Applicant: Shenzhen KangerTech Technology Co., Ltd., Shenzhen (CN)

(72) Inventor: Xiaochun ZHU, Shenzhen (CN)

(21) Appl. No.: 16/886,812

(22) Filed: May 29, 2020

(30) Foreign Application Priority Data
Oct. 23, 2019 (CN) 201911012877 .1

(51) Int. Cl. A24F 15/01; A24F 40/85; A24F 15/18; A24F 40/10; A24F 40/42; A24F 40/46; A24F 40/50; A24F 40/90; A61L 2/26; A61L 2/10

(52) U.S. Cl. CPC A24F 15/01; A24F 40/85; A24F 15/18; A24F 40/10; A24F 40/42; A24F 40/46; A61L 2202/11; A24F 40/90; A61L 2/26; A61L 2/10; A61L 2202/122; A61L 2202/121; A61L 2202/123; A24F 40/50

(57) ABSTRACT

A sterilization box includes a box body with a receiving room for receiving an electronic cigarette therein, a lid including a sterilization device formed on an end towards the receiving room and configured to sterilize the electronic cigarette, and a locking device including a clamping structure, an inserting structure which includes a pressing member, a first spring abutted against the pressing member to push the pressing member to be reset, an inserting member received in and extending out of the receiving room and including a wedge-shaped upper surface to contact with the pressing member which is buckled with the inserting member and then pushing the inserting member to move along a longitudinal direction of the box body for separating from the clamping structure to quickly open the lid and the box body, which can sterilize the electronic cigarette to ensure users' health with a firmly installation and a small footprint thereof.

### CROSS-REFERENCE TO RELATED APPLICATION

This application claims the benefits of CN Application NO. CN 201911012877.1, with Title “STERILIZATION BOX FOR ELECTRONIC CIGARETTE”, filed on Oct. 23, 2019, the disclosure which is incorporated herein by reference.

### BACKGROUND

### Technical Field

The present disclosure generally relates to electronic cigarettes field, and especially relates to a sterilization box for an electronic cigarette and an electronic cigarette assembly thereof.

### Description of Related Art

A common electronic cigarette generally includes a suction member, an atomizing core member and a host. Its working principle is that the atomizing core member is controlled by the host to heat e-liquid received therein to generate gas and then users of the electronic cigarette can suction the gas through the suction member. After the electronic cigarette is used, saliva is often left in the suction member. If the electronic cigarette is stored for a long time in a humid and gas-filled enclosed space, bacteria are likely to grow in the suction member to affect the user's health. Currently, a sterilization box on the market is specially provided for sterilizing the suction member of the electronic cigarette and generally includes a box cover, a box body buckled with the box cover and a locking device fastened the box cover with the box body. The locking device in the related art is generally arranged on surfaces of the box cover and the box body, so that the sterilization box is uneven to affect its appearance. Furthermore, with the increase of the number of times of the electronic cigarette being used, the locking device is prone to wear and loose, which easily leads to a loss and an unstable lock of the locking device. In this way, internal items of the sterilization box can easily fall off to affect a normal use of the sterilization box. Therefore, the conventional technology is needed to be developed.

### SUMMARY

The technical problems to be solved: in view of the shortcomings of the related art, the present disclosure relates to a sterilization box for an electronic cigarette and an electronic cigarette assembly thereof which can effectively sterilize the electronic cigarette to ensure the health of users, and as well as a firmly installation and a small footprint thereof.

The technical solution adopted for solving technical problems of the present disclosure is:

a sterilization box for an electronic cigarette includes a box body including a receiving room for receiving the electronic cigarette therein, a lid including a sterilizing device arranged at one end thereof towards the receiving room and configured to sterilize the electronic cigarette, and a locking device configured to lock the lid and the box body. The locking device includes an inserting structure arranged on the box body, a clamping structure embedded in the lid and used for inserting into and clamping with the inserting structure; the inserting structure including a pressing member, a first spring, and an inserting member received in the box body and extending out of the box body, with a wedge-shaped upper surface thereof contacting with the pressing member. The pressing member is buckled with the inserting member and then pushing the inserting member to move along a longitudinal direction of the box body so that the inserting member is separated from the clamping structure to quickly open the lid and the box body. The first spring is abutted against the pressing member to push the pressing member back to an original position of the pressing member.

An electronic cigarette assembly according to an embodiment of the present disclosure includes a sterilization box and an electronic cigarette received in the sterilization box. The sterilization box includes a box body including a receiving room for receiving the electronic cigarette therein, a lid including a sterilizing device arranged at one end thereof towards the receiving room and configured to sterilize the electronic cigarette, and a locking device configured to lock the lid and the box body. The locking device includes an inserting structure arranged on the box body, a clamping structure embedded in the lid and used for inserting into and clamping with the inserting structure; the inserting structure including a pressing member, a first spring, and an inserting member received in the box body and extending out of the box body, with a wedge-shaped upper surface thereof contacting with the pressing member. The pressing member is buckled with the inserting member and then pushing the inserting member to move along a longitudinal direction of the box body so that the inserting member is separated from the clamping structure to quickly open the lid and the box body. The first spring is abutted against the pressing member to push the pressing member back to an original position of the pressing member. The electronic cigarette includes a suction member, an atomizing core member, a base member and a host connected successively from top to bottom. The suction member is abutted against the host via an outer wall thereof and includes an e-liquid storage tank; the base member buckled and fixed with the suction member for sealing the e-liquid storage tank. The electronic cigarette further includes a tapered slope extending longitudinally from a top surface of the electronic cigarette through a sidewall of the electronic cigarette to a bottom surface of the electronic cigarette along a longitudinal direction of the electronic cigarette.

The present disclosure provides the advantages as below.

The structure of the present disclosure can provide a box body including a receiving room for receiving the electronic cigarette therein, a lid including a sterilizing device arranged at one end thereof towards the receiving room and configured to sterilize the electronic cigarette, so that a surface of the electronic cigarette can always be kept clean and hygienic, so as to ensure the health of users using the electronic cigarette. At the same time, the structure of the present disclosure can further provide an inserting structure arranged on the box body, a clamping structure embedded in the lid and used for inserting into and clamping with the inserting structure, and an inserting member received in the box body and extending out of the box body, so that the locking device can integrally and stably fixed between the lid and the box body, which is convenient to use and carry, occupies a small volume to difficultly drop and lose. Meanwhile, a wedge-shaped upper surface contacted with the pressing member is provided on the inserting member, and the pressing member is buckled with the inserting member and then pushed the inserting member to move along a longitudinal direction of the box body so that the inserting member can be separated from the clamping structure so as to quickly open the lid and the box body, which can be convenient to use.

### BRIEF DESCRIPTION OF THE DRAWINGS

FIG. 1 is a schematic view of a sterilization box in accordance with an embodiment of the present disclosure.

FIG. 2 is an exploded, schematic view of the sterilization box of FIG. 1.

FIG. 3 is another schematic view of the sterilization box of FIG. 1.

FIG. 4 is a further exploded, schematic view of the sterilization box of FIG. 2.

FIG. 5 is an enlarged view of the circle IV of the sterilization box of FIG. 4.

FIG. 6 is a side cross-sectional view of the sterilization box of the present disclosure.

FIG. 7 is an enlarged view of the circle VI of the sterilization box of FIG. 6.

FIG. 8 is a schematic view of an inserting member of the sterilization box of the present disclosure.

FIG. 9 is an exploded, schematic view of the inserting member of the sterilization box of FIG. 8.

FIG. 10 is a schematic view of a pressing member of the sterilization box of the present disclosure.

FIG. 11 is a schematic view of a second spring of the sterilization box of the present disclosure.

FIG. 12 is a schematic view of the sterilization box received in an electronic cigarette of the present disclosure.

FIG. 13 is a schematic view of a sterilizing device of the sterilization box of the present disclosure.

FIG. 14 is a schematic view of an electronic cigarette assembly of the present disclosure.

FIG. 15 is an exploded, schematic view of the electronic cigarette assembly of FIG. 14.

FIG. 16 is a schematic view of the electronic cigarette of the electronic cigarette assembly of the present disclosure.

FIG. 17 is an exploded, schematic view of the electronic cigarette of the electronic cigarette assembly of the present disclosure.

FIG. 18 is an exploded, schematic view of a suction member of the electronic cigarette assembly of the present disclosure.

FIG. 19 is an exploded, schematic view of a host of the electronic cigarette assembly of the present disclosure.

FIG. 20 is a schematic view of an e-liquid feeding base and a pair of gaskets of the electronic cigarette assembly of the present disclosure.

FIG. 21 is a cross-sectional view of the suction member of the electronic cigarette assembly of the present disclosure.

FIG. 22 is cross-sectional view of the host of the electronic cigarette assembly of the present disclosure.

### DETAILED DESCRIPTION

Reference will now be made in detail to embodiments, examples of which are illustrated in the accompanying drawings. In the following detailed description, numerous specific details are set forth in order to provide a thorough understanding of the subject matter presented herein. Obviously, the implementation embodiment in the description is a part of the present disclosure implementation examples, rather than the implementation of all embodiments, examples. According to the described embodiment of the present disclosure, all other embodiments obtained by one of ordinary skill in the related art on the premise of no creative work are within the protection scope of the present disclosure.

In the description of the present disclosure, it needs to be explained that all the directional indicators (such as the terms: “upper”, “below”, “left”, “right”, “front”, “back” . . . ), are shown in the specification of the present disclosure. The indicated orientation or position of the terms shown in the detailed description is based on the orientation or position shown in the figures of the accompanying drawings of the present disclosure, which is only to easily simplify the description of the present disclosure, but not indicated that the devices or elements of the present disclosure should have a particular orientation or should be designed and operated in a particular orientation. So the terms illustrated in the detail description are not by way of the limitation of the present disclosure.

In the description of the present disclosure, except where specifically otherwise illustrated or limited, the terms “connect” and “link” used herein should be understood in a broad sense. Such as, the meaning may be tight connection, removable connection, or integrated connection. The meaning may also be mechanical connection, electrical connection, direct connection or indirect connection through intermediaries, or internal connection within two elements. The meaning of the terms used herein may be understood by one of ordinary skill in the related art according to specific conditions of the present disclosure.

Furthermore, in the description of the present disclosure, the terms such as “first” and “second” shown in the specification are only used to describe, but not indicated that the elements of the present disclosure is important or represented the amount of the elements. That is, the features limited by the terms of “first” and “second” may explicitly or implicitly include one or more features.

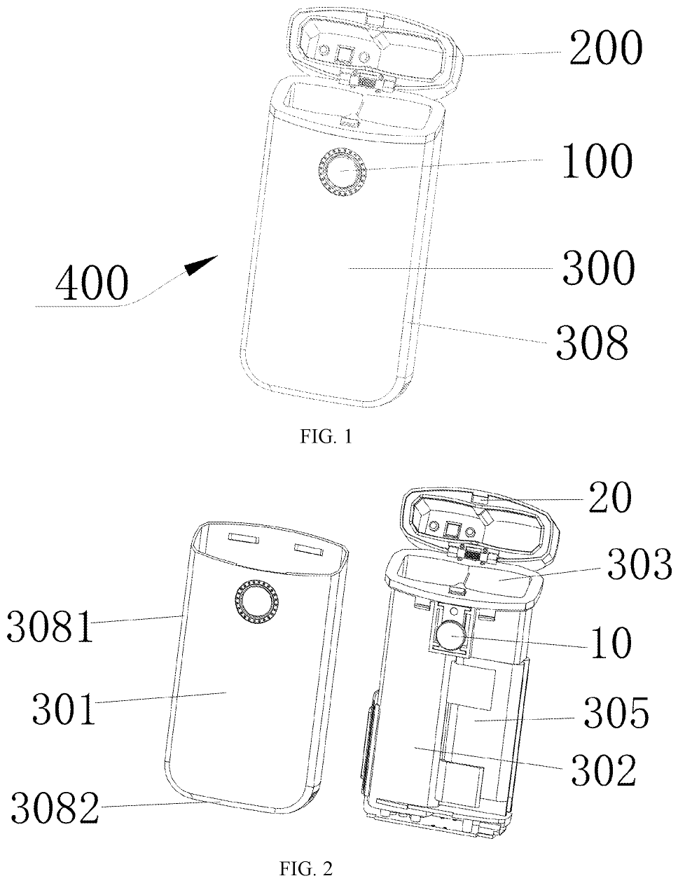

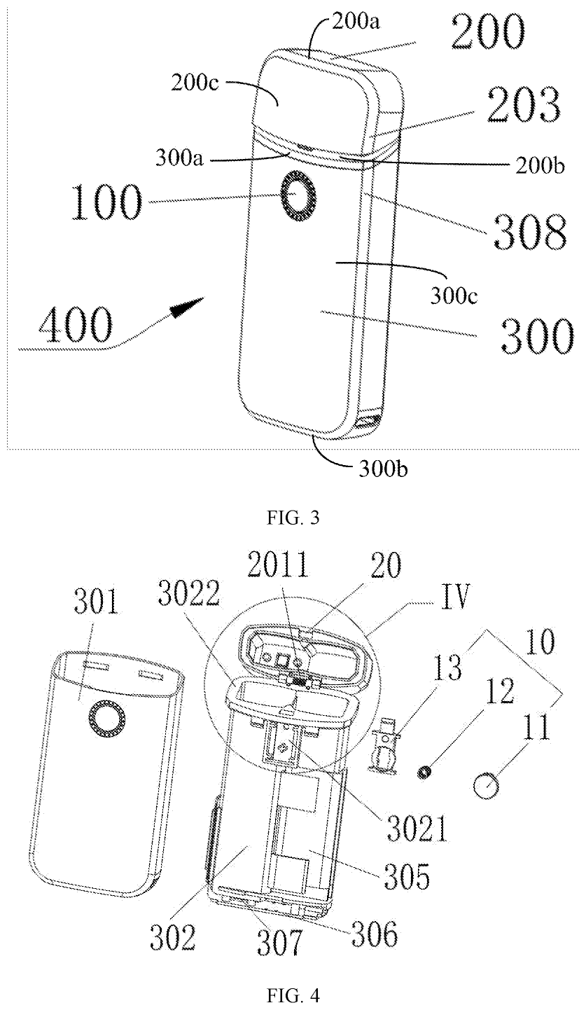

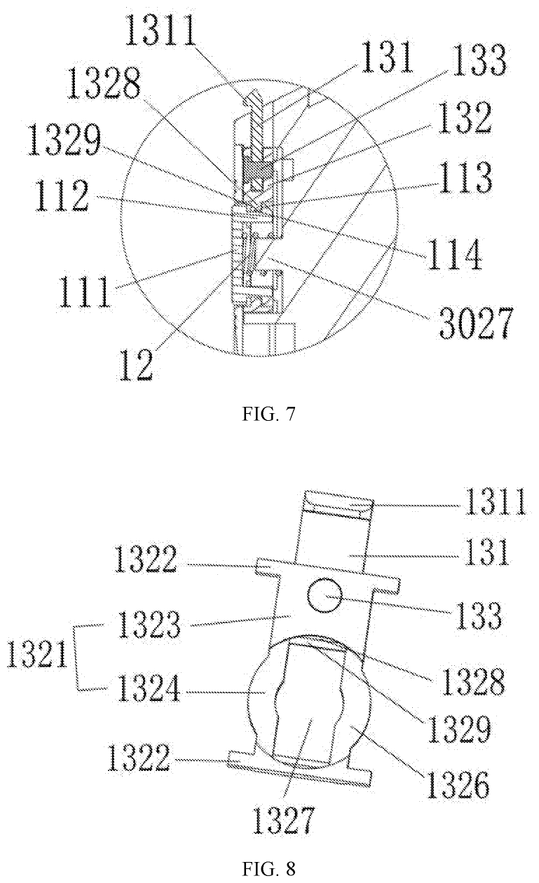

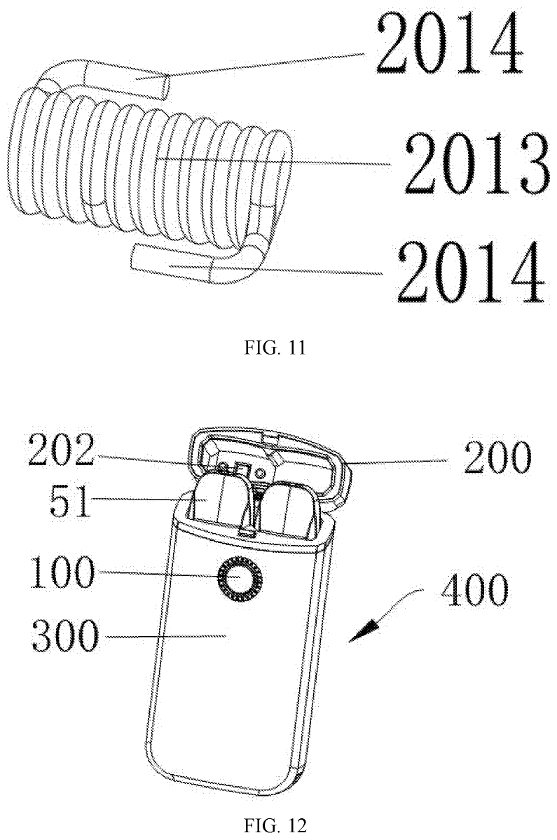

Referring to FIGS. 1-13, a sterilization box 400 used for an electronic cigarette 500 according to an embodiment of the present disclosure includes a lid 200, a box body 300, and a locking device 100 fastened the lid 200 and the box body 300. The box body 300 includes a receiving room 303 for receiving the electronic cigarette 500 therein, and the lid 200 includes a sterilizing device 202 arranged at one end thereof towards the receiving room 303 and configured to sterilize the electronic cigarette 500. The locking device 100 includes an inserting structure 10 arranged on the box body 300, and a clamping structure 20 embedded in the lid 200 and used for inserting into and clamping with the inserting structure 10. The inserting structure 10 includes a pressing member 11, a first spring 12, and an inserting member 13 received in the box body 300 and extending out of the box body 300 so that the locking device 100 can integrally and stably fixed between the lid 200 and the box body 300, which is convenient to use and carry, occupies a small volume to difficultly drop and lose. A wedge-shaped upper surface 1329 contacted with the pressing member 11 is provided on the inserting member 13, and the pressing member 11 is buckled with the inserting member 13 and then pushed the inserting member 13 to move along a longitudinal direction of the box body 300 so that the inserting member 13 can be separated from the clamping structure 20 so as to quickly open the lid 200 and the box body 300. The first spring 12 is abutted against the pressing member 11 to push the pressing member 11 back to an original position of the pressing member 11 for a next reuse.

During using the sterilization box 400, a side of the lid 200 is directly flip towards the inserting structure 10, and then the inserting structure 10 can enter the clamping structure 20 and be fixed with the clamping structure 20, thus, the lid 200 is locket with the box body 300. When the lid 200 is needed to be opened, the pressing member 11 is pressed and then moved along a transverse direction of the box body 300, at this time, the pressing member 11 is in contact with the wedge-shaped surface 1329 of the inserting member 13 and then pushes the inserting member 13 to move along the longitudinal direction of the box body 300. When the inserting member 13 is moved along the longitudinal direction of the box body 300 until it is detached from the clamping structure 20, the lid 200 and the box body 300 can be opened, which is convenient and quick.

The sterilization box 400 used for the electronic cigarette 500 of the present disclosure can provide the box body 300 including the receiving room 303 for receiving the electronic cigarette 500 therein, the lid 200 including the sterilizing device 202 arranged at one end thereof towards the receiving room 303 and configured to sterilize the electronic cigarette 500, so that a surface of the electronic cigarette 500 can always be kept clean and hygienic, so as to ensure the health of users using the electronic cigarette 500. At the same time, the structure of the present disclosure can further provide the inserting structure 10 arranged on the box body 300, the clamping structure 20 embedded in the lid 200 and used for inserting into and clamping with the inserting structure 10, and the inserting member 13 received in the box body 300 and extending out of the box body 300, so that the locking device 100 can integrally and stably fixed between the lid 200 and the box body 300, which is convenient to use and carry, occupies a small volume to difficultly drop and lose. Meanwhile, the wedge-shaped upper surface 1329 contacted with the pressing member 11 is provided on the inserting member 13, and the pressing member 11 is buckled with the inserting member 13 to push the inserting member 13 to move along the longitudinal direction of the box body 300 so that the inserting member 13 is separated from the clamping structure 20 to quickly open the lid 200 and the box body 300, which is convenient to use.

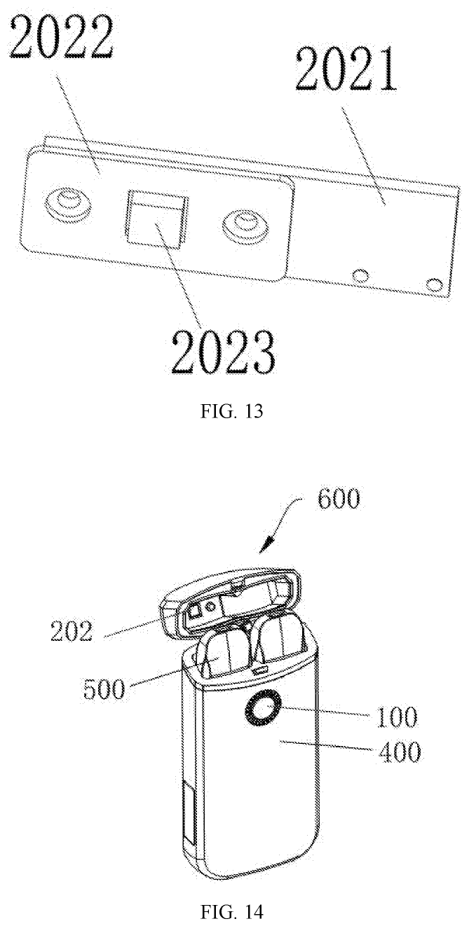

Referring to FIG. 13, in an embodiment of the present disclosure, the sterilizing device 202 further includes a sterilizing plate 2021 and a sealing gasket 2022. The sterilizing plate 2021 includes an ultraviolet lamp 2023 and the sealing gasket 2022 is clamped between the sterilizing plate 2021 and the box body 300 to prevent the sterilizing plate 2021 from being damaged.

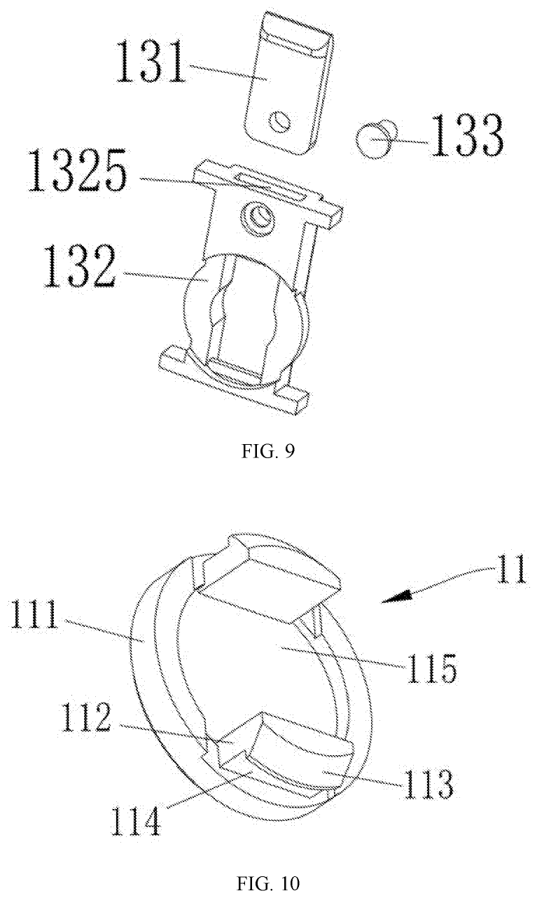

Referring to FIG. 4, FIG. 8 and FIG. 9, the inserting member 13 includes a lock catch 131 and a sliding block 132 received in the box body 300. One end of the lock catch 131 is tightly fixed with the sliding block 132 by a mounting member 133. In an embodiment of the present disclosure, the mounting member 133 is a dowel, in other embodiments of the present disclosure, the lock catch 131 can be integrated with the sliding block 132 to form an integral structure as long as the lock catch 131 is ensured to move together with the sliding block 132. The other end of the lock catch 131 is extended out of the box body 300 along the longitudinal direction of the box body 300, and extended laterally at a distal end of the lock catch 131 to form a first barb 1311, the first barb 1311 extending into the clamping structure 20 to clamp the lid 200 with the box body 300.

Preferably, the box body 300 includes a housing 301 and a holder 302 fixed in the housing 301. A receiving recess 3021 is formed on a surface of the holder 302 towards the housing 301 to receive the sliding block 132 therein, so as to conveniently receive and assembly the sliding block 132. A connecting step 3022 is formed on the top of the holder 302, with a first through-hole thereof being communicated with the receiving recess 3023, and the lock catch 131 is extended out of the box body 300 through the first through-hole 3023. A contact surface of the lid 200 connected with the box body 300 is a surface of the connecting step 3022, in an embodiment of the present disclosure, the surface of the connecting step 3022 is inclined, which can increase a contact area between the lid 200 and the box body 300 to reduce stress generation therebetween, and is conducive to improve a locking stability between the lid 200 and the box body 300.

Specifically, the sliding block 132 includes a main body 1321 and a pair of beams 1322 respectively arranged on two opposite ends of the main body 1321. In an embodiment of the present disclosure, the configuration of the sliding block 132 is an H-shaped structure after being turned around 90 degree, so as to ensure sliding stability of the sliding block 132. The receiving recess 3021 includes a sliding recess 3024 corresponding to the main body 1321 so that the main body 1321 can slide in the sliding recess 3024, and a limiting recess 3025 corresponding to the pair of beams 1322 for limiting a movement range of the beam 1322 to prevent the pair of beams 1322 from disengaging from the limiting recess 3025. A sidewall of the receiving recess 3021 is provided with two pairs of locating posts 3026 arranged opposite to each other. The two pairs of locating posts 3026 and a top wall of the receiving recess 3021 or the two pairs of locating posts 3026 and a bottom wall of the receiving recess 3021 are respectively enclosed into the limiting recess 3025, a width of the limiting recess 3025 along the longitudinal direction of the box body 300 is greater than a thickness of the beam 1322 along longitudinal direction of the box body 300 to ensure that the beam 1322 can move smoothly in the limiting recess 3025.

Referring to FIG. 8 and FIG. 9, the main body 1321 includes a connecting portion 1323 and a fastening portion 1324. The connecting portion 1323 includes a longitudinal installing groove 1325 along the transverse direction of the box body 300 so that the lock catch 131 can insert into the installing groove 1325 to be fastened in the installing groove 1325. The fastening portion 1324 includes a concave portion 1326, a locating slot 1327 passing through the concave portion 1326. A height of the concave portion 1326 is lower than that of the connecting portion 1323, which is conducive to reduce an area occupied by the pressing member 11 and improve compactness of each elements therebetween when the pressing member 11 is buckled with the concave portion 1326. In addition, two sides of the concave portion 1326 extend outward to form a circular arc structure to further prevent the sliding block 132 from being detached from the receiving recess 3021. A pair of wedge-shaped blocks 1328 is arranged on two opposite ends of the locating slot 1327, and the wedge-shaped surface 1329 is formed on the wedge-shaped block 1328.

Referring to FIGS. 6-7 and FIG. 10, the pressing member 11 includes a pressing plate 111, a pair of enclosing walls 112 extending inwardly from an edge of the pressing plate 111 and formed opposite to each other, and a second barb 113 formed by a distal end of the enclosing wall 112 extending outwardly. All the pressure plate 111, the enclosing wall 112 and the second barb 113 are cooperatively enclosed to form a recess 114 for receiving the wedge-shaped block 1328 therein. The second barb 113 can pass through the locating slot 1327 and then clamp with the wedge-shaped block 1328 to connect the pressing member 11 with the sliding block 132. When the pressing plate 111 is pressed to move along the transverse direction of the box body 300, the pressing plate 111 can connect with the wedge-shaped block 1328 and then push the wedge-shaped block 1328 to move along longitudinal direction of the box body 300. Because the wedge-shaped block 1328 is fixed with the sliding block 132 and the sliding block 132 is fixed with the lock catch 131, both the sliding block 132 and the lock catch 131 are driven to follow moving along longitudinal direction of the box body 300. At this time, the second barb 1311 is also moved upwardly along the longitudinal direction of the box body 300 until it is disengaged from the clamping structure 20, thereby the lid 200 can be opened. Due to provide the wedge-shaped block 1328, when the pressing plate 111 is moved along the transverse direction of the box body 300, the lock catch 131 and the sliding block 132 can also move along the longitudinal direction of the box body 300.

Preferably, a cavity 115 is surrounded by the pair of enclosing walls 112 for receiving the first spring 12 therein, and a surface of the holder 302 towards the housing 301 is extended upwardly to form a protrusion 3027 thereof. One end of the first spring 12 is sleeved around the protrusion 3027, and the other opposite end of the first spring 12 is abutted against the pressing plate 111. When pressing the pressing plate 111, the first spring 12 is in a compressed state. After the lid 200 and the box body 300 are opened, the pressing plate 111 is released and the first spring 12 is reset to push the pressing plate 111 to reset for a convenient next use.

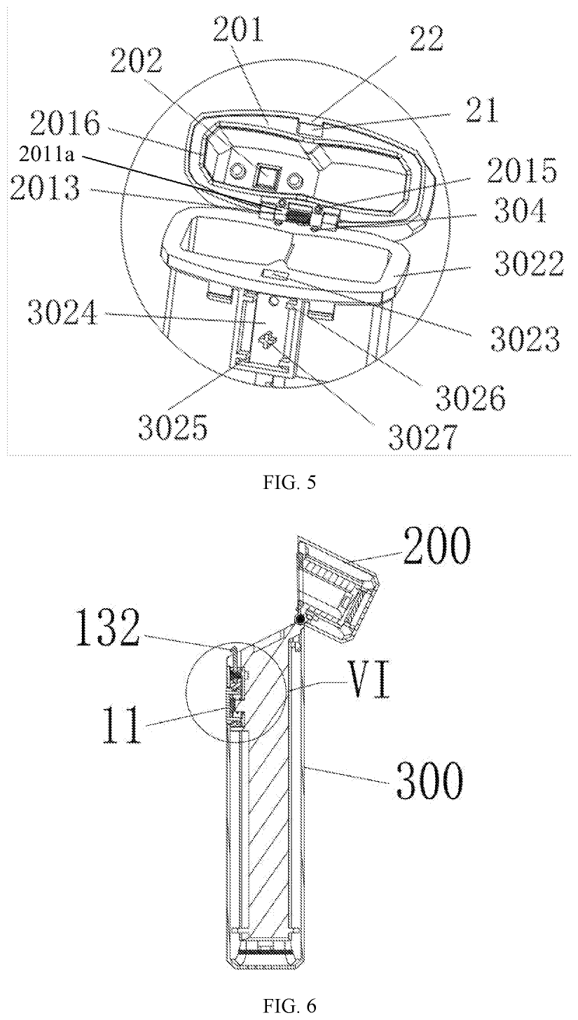

Furthermore, referring to FIG. 5, the clamping structure 20 includes a catching groove 21 and a hook 22 arranged on an edge of the catching groove 21. Both the hook 22 and the first barb 1311 are arranged along opposite directions, the lock catch 131 is extended into the catching groove 21 so that both the first barb 1311 and the hook 22 are fastened together to tightly fix the lid 20 with the box body 300.

Specifically, an inner lining 201 is arranged on the lid 200 and the sterilizing device 202 is arranged on the inner lining 201. A first sealing ring 2016 is arranged at one end of the inner lining 201 close to the box body 300. When the sterilization box 400 is closed, the electronic cigarette 500 is completely sealed to completely isolate from outer environments, so as to prevent the suction member 501 from being polluted, and prevent the e-liquid leakage of the electronic cigarette 500.

Referring to FIG. 4 and FIG. 11, preferably, the inner lining 201 includes a hinge 2011 formed on an edge of one side thereof and hinged to a side of the box body 300 via a rotating shaft 2011a. The hinge 2011 includes a second spring 2013 formed therein and passing through the rotating shaft 2011a, and a pair of bending portions 2014 formed by two ends of the second spring 2013 and extending reversely and in parallel. One bending portion 2014 is abutted against the inner lining 201 and the other bending portion 2014 is abutted against a top surface 300a of the box body 300. When the lid 200 is locked with the box body 300, the second spring 2013 is in a compressed state. When the lid 200 and the box body 300 are unlocked, an elastic force of the second spring 2013 is applied for driving the lid 200 to automatically flip around the rotation shaft 2011a to open the lid 200, which is convenient and quick to be operated with one hand.

Referring to FIG. 4 and FIG. 5, a first electrode 2015 is arranged on the inner lining 201 close to the hinge 2011 and electrically connected to the sterilizing device 202, and a second electrode 304 is arranged on the top surface 300a of the box body 300 and corresponding to the first electrode 2015. The box body 300 further includes a first battery 305, a first control circuit board 306 and a charging electrode 307 arranged on the bottom of the receiving room 303. All of the second electrode 304, the first control circuit board 306 and the charging electrode 307 are electrically connected to the first battery 305, and the first electrode 2015 is connected with the second electrode 304 to drive the sterilizing device 202 to work. When the electronic cigarette 500 is received in the receiving room 303, the charging electrode 307 is electrically connected with a corresponding electrode at the bottom of the electronic cigarette 500 to charge the electronic cigarette 500. When the electronic cigarette 500 is received in the receiving room 303 and the lid 200 is locked with the box body 300, at this time, the first electrode 2015 is in contact with the second electrode 304, and the electronic cigarette 500 is detected and recognized by the first control circuit board 306, and then the sterilizing device 202 is driven to power on, in order to seal and sterilize the suction member 51 of the electronic cigarette 500. If the first control circuit board 306 is detected that the lid 200 is not covered, it is only to charge the electronic cigarette 500. If the electronic cigarette 500 isn't placed into the receiving room 30, the electronic cigarette 500 can't be recognized by the first control circuit board 306, so the sterilization device 202 isn't powered on, thereby no sterilization operation is produced.

Referring to FIGS. 1-3, a U-shaped slope 308 is formed between the top surface 300a of the box body 300 and a periphery sidewall 300c of the box body 300, and between a bottom surface 300b of the box body 300 and the periphery sidewall 300c of the box body 300, respectively. The U-shaped slope 308 is arranged with ergonomic and aesthetics.

Preferably, the U-shaped slope 308 includes a pair of longitudinal slopes 3081 formed in parallel and opposite to each other, and a transverse slope 3082 connected with the pair of longitudinal slopes 3081. An arc structure is formed on a connection of the longitudinal slope 3081 and the transverse slope 3082. In this way, the longitudinal slope 3081 and the transverse slope 3082 can be connected smoothly and roundness, which can effectively solve the visual violation and impact caused by a right angle design, and improve the hard and impact of a rectangular box designed in the related art.

Specifically, an inverted U-shaped slope 203 is formed between an upper surface 200a of the lid and a lateral sidewall 200c of the lid 200, and between a lower surface 200b of the lid 200 and the lateral sidewall 200c of the lid 200, respectively. When the lid 200 is covered on the box body 300, the inverted U-shaped slope 203 is connected with the U-shaped slope 308 to form an overall consistent inclined plane structure, which is clean and beautiful.

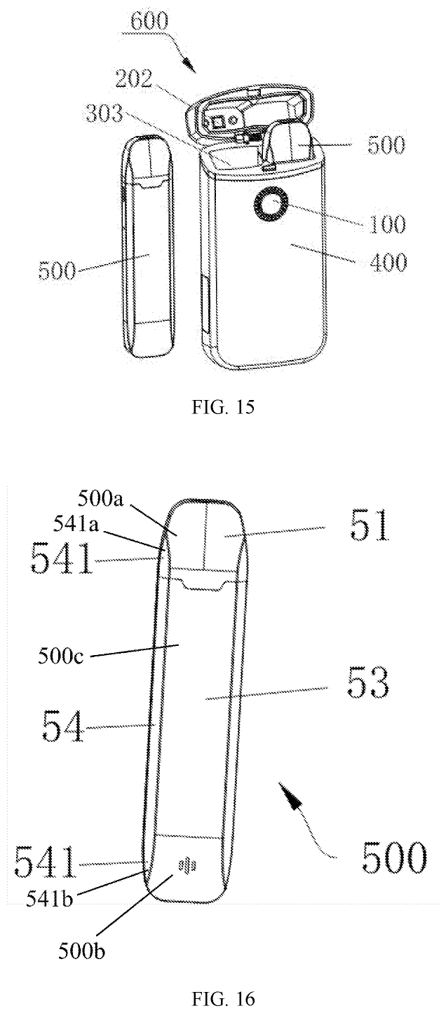

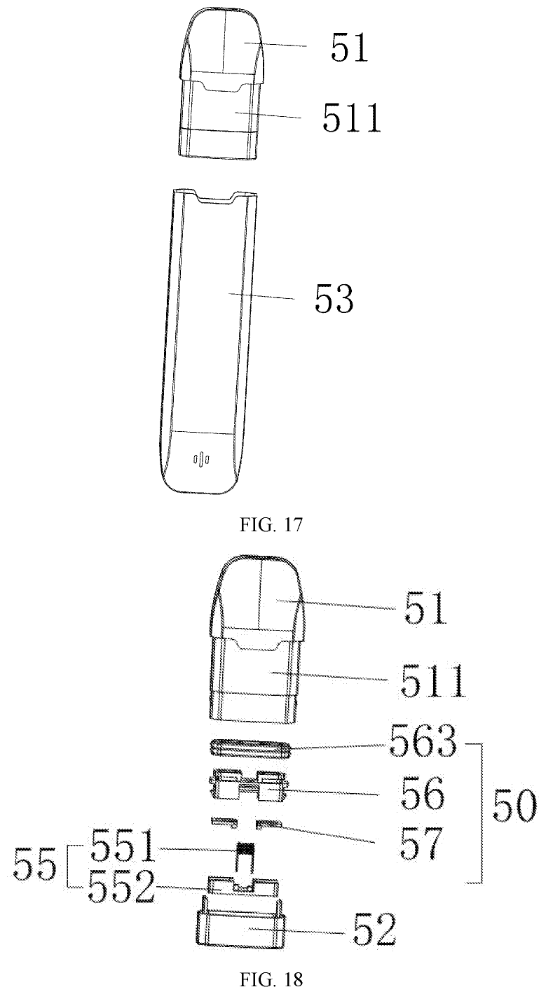

Referring to FIGS. 14-18, an electronic cigarette assembly 600 according to an embodiment of the present disclosure includes a sterilization box 400 and an electronic cigarette 500 received in the sterilization box 400. The sterilization box 400 includes a lid 200, a box body 300, and a locking device 100 fastened the lid 200 and the box body 300. The box body 300 includes a receiving room 303 for receiving the electronic cigarette 500 therein, and the lid 200 includes a sterilizing device 202 arranged at one end thereof towards the receiving room 303 and configured to sterilize the electronic cigarette 500. The locking device 100 includes an inserting structure 10 arranged on the box body 300, and a clamping structure 20 embedded in the lid 200 and used for inserting into and clamping with the inserting structure 10. The inserting structure 10 includes a pressing member 11, a first spring 12, and an inserting member 13 received in the box body 300 and extending out of the box body 300. A wedge-shaped upper surface 1329 contacted with the pressing member 11 is provided on the inserting member 13, and the pressing member 11 is buckled with the inserting member 13 and then pushed the inserting member 13 to move along a longitudinal direction of the box body 300 so that the inserting member 13 can be separated from the clamping structure 20 so as to quickly open the lid 200 and the box body 300. The first spring 12 is abutted against the pressing member 11 to push the pressing member 11 back to an original position of the pressing member 11 for a next reuse.

The locking device 100 of the electronic cigarette assembly 600 of the present disclosure is provided for integrally and stably fixing between the lid 200 and the box body 300, which is convenient to use and carry, occupies a small volume to difficultly drop and lose.

The electronic cigarette 500 of the present disclosure includes a suction member 51, an atomizing core member 50, a base member 52 and a host 53 connected successively from top to bottom. The suction member 51 is abutted against the host 53 via an outer wall thereof and includes an e-liquid storage tank 511. The base member 52 is buckled and fixed with the suction member 51 for sealing the e-liquid storage tank 511. The electronic cigarette 500 further includes a tapered slope 54 extending longitudinally from a top surface 500a of the electronic cigarette 500 through a sidewall 500c of the electronic cigarette 500 to a bottom surface 500b of the electronic cigarette 500 along a longitudinal direction of the electronic cigarette 500.

The electronic cigarette 500 of the present disclosure is provided that the suction member 51 is abutted against the host 53 via an outer wall thereof and includes the e-liquid storage tank 511, and the base member 52 is buckled and fixed with the suction member 51 for sealing the e-liquid storage tank 511. That is, the suction member 51 is integrated with the e-liquid storage tank 511 to form an integral structure for conveniently and stably installing the suction member 51 on the atomizing core member 50. The electronic cigarette 500 further includes the tapered slope 54 extending longitudinally from the top surface 500a of the electronic cigarette 500 through the sidewall 500c of the electronic cigarette 500 to the bottom surface 500b of the electronic cigarette 500 along the longitudinal direction of the electronic cigarette 500, which is ergonomic and more conducive to take and increase the appearance by a streamlined design.

Furthermore, the tapered slope 54 is extended from a middle position of the host 53 to both ends of the host 53 along the longitudinal direction of the electronic cigarette 500, and then gradually folded and aggregated at a distal end of the electronic cigarette 500 to form a tapered portion 541. One end 541a of the tapered portion 541 is extended to the suction member 51 and the other opposite end 541b of the tapered portion 541 is extended to the bottom of the host 53.

Preferably, the atomizing core member 50 includes a heating member 55 which includes a heating wire 551 and a silicone case 552. The heating wire 551 is configured to heat and atomize heated e-liquid in the e-liquid storage tank 511 to generate gas for being sucked by users of the electronic cigarette 500. The heating wire 551 passes through the silicone case 552 and then is fixed with the silicone case 552. The silicone case 552 is embedded in the base member 52 to separate the base member 52 from the e-liquid storage tank 511 to prevent the heated e-liquid in the e-liquid storage tank 511 from permeating into the base member 52.

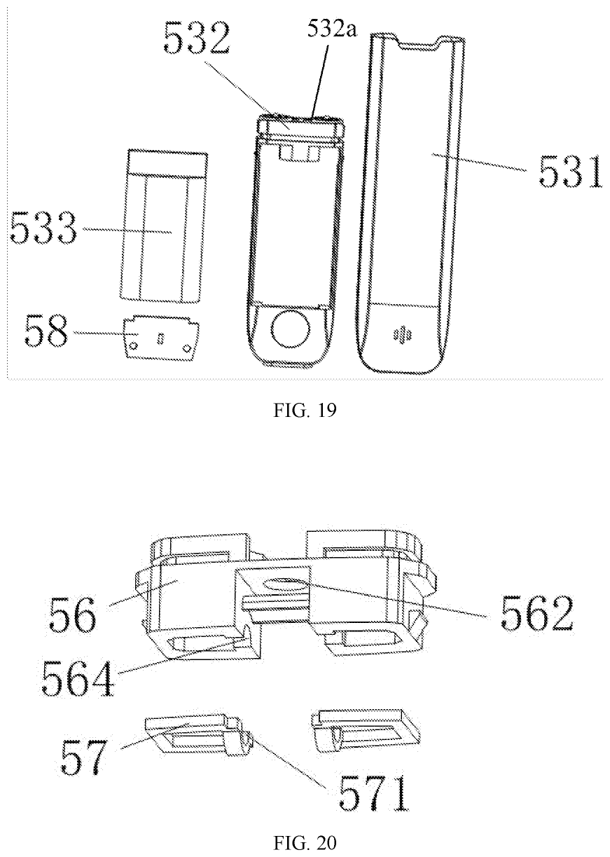

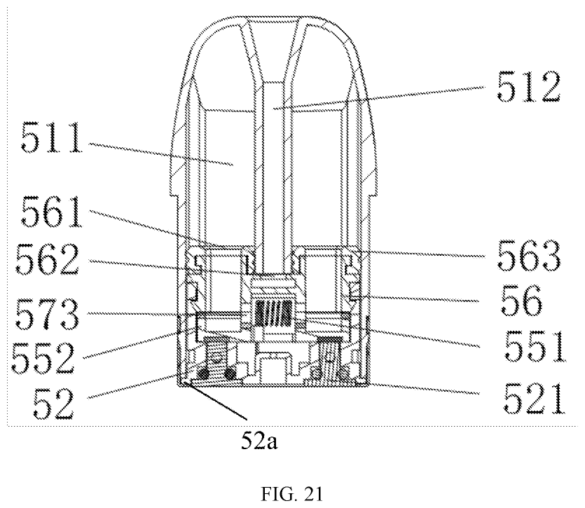

Referring to FIGS. 18-21, the atomizing core member 50 further includes an e-liquid feeding base 56 buckled with the base member 52 and abutted against the silicone case 552. The e-liquid feeding base 56 includes a pair of longitudinal e-liquid feeding holes 561 connected with the e-liquid storage tank 511 so as to conveniently remove the suction member 51 and then add the heated e-liquid into the e-liquid feeding base 56.

Specifically, a pair of gaskets 57 is arranged between the e-liquid feeding base 56 and the silicone base 552 and configured to increase a tight connection between the e-liquid feeding base 56 and the silicone base 552. The pair of gaskets 57 is respectively embedded on the silicone base 552, and each of the pair of gaskets 57 includes a first semicircular groove 571. The e-liquid feeding base 56 includes a second semicircular groove 564 formed on the bottom thereof and corresponding to the first semicircular groove 571. Each first semicircular groove 571 and each second semicircular groove 564 are cooperatively enclosed to form an opening 573 therebtween so that the heating wire 551 is formed between the pair of openings 573. In this way, the heated e-liquid in the e-liquid storage tank 511 can enter the atomizing core member 50 and the heating wire 551 through the pair of openings 573.

Preferably, an air passage 512 is formed on the suction member 51 along the longitudinal direction of the electronic cigarette 500 to divide the e-liquid storage tank 511 into two halves. The e-liquid feeding base 56 includes a second through-hole 562 connected with the air passage 512. In this way, the heated e-liquid in the e-liquid storage tank 511 is heated and atomized by the atomizing core member 51 to generate the gas, and then the gas is discharged by the air passage 512 to be used by the user of the electronic cigarette 500.

Furthermore, a second sealing ring 563 is arranged on the e-liquid feeding base 56. An outer wall of the second sealing ring 563 is abutted against an inner wall of the suction member 51, and an inner wall of the second sealing ring 563 is abutted against the e-liquid feeding base 56 and the air passage 512, respectively, to prevent the heated liquid from permeating to other places and reduce the pollution to other components..

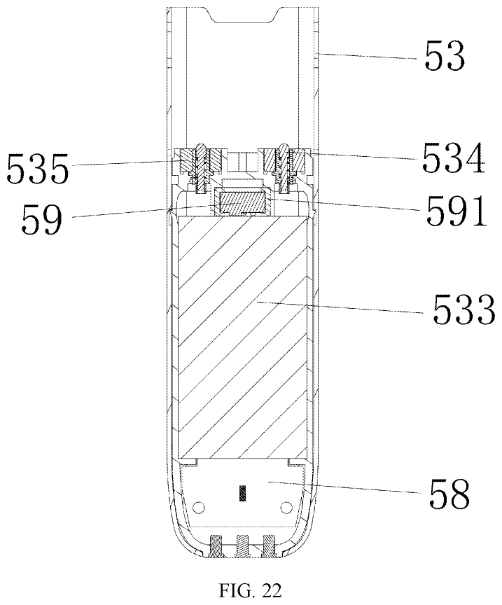

Referring to FIG. 18 and FIG. 22, the host 53 includes a case 531 and a frame 532 fixed in the case 531 and receiving a second battery 533 therein. An upper end surface 532a of the frame 532 is abutted against a bottom end surface 52a of the base member 52, a third electrode 521 is embedded in the bottom end surface 52a of the base member 52 and electrically connected with the atomizing core member 50, and a fourth electrode 534 is embedded in the upper end surface 532a of the frame 532 and electrically connected with the second battery 533. The third electrode 521 is electrically connected with the fourth electrode 534 so as to supply power for the heating member 55 and the atomizing core member 50.

Preferably, a magnet 535 is surrounded around the fourth electrode 534 and then attractably fixed with the third electrode 521 to stably connect the base member 52 with the host 53.

Preferably, the electronic cigarette 500 further includes a second control circuit board 58 positioned below the second battery 533 and electrically connected to the second battery 533, a microphone 59 electrically connected to the second control circuit board 58, and a microphone silicone base 591 sleeved around the microphone 59 to prevent the microphone 59 from being contaminated. When the suction member 51 is suctioned, the microphone 59 is configured to drive the heating wire 551 to work.

Although the features and elements of the present disclosure are described as embodiments in particular combinations, each feature or element can be used alone or in other various combinations within the principles of the present disclosure to the full extent indicated by the broad general meaning of the terms in which the appended claims are expressed.

What is claimed is:

1. A sterilization box for an electronic cigarette comprising:
a box body comprising a receiving room for receiving the electronic cigarette therein;
a lid comprising a sterilizing device arranged at one end thereof towards the receiving room and configured to sterilize the electronic cigarette;
a locking device configured to lock the lid and the box body and comprising an inserting structure arranged in the box body, a clamping structure embedded in the lid and used for inserting into and clamping with the inserting structure; the inserting structure comprising a pressing member, a first spring, and an inserting member received in the box body and extending out of the box body, with a wedge-shaped upper surface thereof contacting with the pressing member; and wherein
the pressing member is buckled with the inserting member and then pushed the inserting member to move along a longitudinal direction of the box body so that the inserting member is separated from the clamping structure to quickly open the lid and the box body; and the first spring is abutted against the pressing member to push the pressing member back to an original position of the pressing member.

2. The sterilization box as claimed in claim 1, wherein the inserting member comprises a lock catch and a sliding block received in the box body, one end of the lock catch tightly fixed with the sliding block by a mounting member, the other end of the lock catch extending out of the box body along the longitudinal direction of the box body, with a distal end thereof extending laterally to form a first barb, the first barb extending into the clamping structure to clamp the lid with the box body.

3. The sterilization box as claimed in claim 2, wherein the box body comprises a housing and a holder fixed in the housing, a receiving recess formed on a surface of the holder towards the housing to receive the sliding block therein, and a connecting step formed on the top of the holder, with a first through-hole thereof being communicated with the receiving recess, and the lock catch extending out of the box body through the first through-hole.

4. The sterilization box as claimed in claim 3, wherein the sliding block comprises a main body and a pair of beams respectively arranged on two opposite ends of the main body, the receiving recess comprising a sliding recess corresponding to the main body for the main body sliding in the sliding recess, a limiting recess corresponding to the pair of beams, and two pairs of locating posts formed on a side wall thereof and opposite to each other so that the limiting recess is formed by the two pairs of locating posts and a top wall or a bottom wall of the receiving recess, a width of the limiting recess along the longitudinal direction of the box body being greater than a thickness of the beam along longitudinal direction of the box body.

5. The sterilization box as claimed in claim 4, wherein the main body comprises a connecting portion comprising a longitudinal installing groove formed along a transverse direction of the box body so that the lock catch can insert into the installing groove to be fastened in the installing groove, and a fastening portion comprising a concave portion, a locating slot passing through the concave portion, and a pair of wedge-shaped blocks arranged on two opposite ends of the locating slot, the wedge-shaped surface formed on the wedge-shaped block; the pressing member comprising a pressing plate, a pair of enclosing walls extending inwardly from an edge of the pressing plate and formed opposite to each other, and a second barb formed by a distal end of the enclosing wall extending outwardly; all of the pressure plate, the enclosing wall and the second barb cooperatively enclosed to form a recess for receiving the wedge-shaped block therein, the second barb passing through the locating slot and then clamped with the wedge-shaped block, the pressing plate configured to push the wedge-shaped block to move along longitudinal direction of the box body so as to drive both the sliding block and the lock catch to follow moving along the longitudinal direction of the box body, when the pressing plate is pressed to move along a transverse direction of the box body.

6. The sterilization box as claimed in claim 5, wherein a cavity is surrounded by the pair of enclosing walls for receiving the first spring therein, a surface of the holder towards the housing extending upwardly to form a protrusion thereof, one end of the first spring sleeved around the protrusion, and the other opposite end of the first spring abutted against the pressing plate.

7. The sterilization box as claimed in claim 2, wherein the clamping structure comprises a catching groove and a hook arranged on an edge of the catching groove, both the hook and the first barb arranged on opposite directions, the lock catch extending into the catching groove so that both the first barb and the hook are fastened together to tightly fix the lid with the box body.

8. The sterilization box as claimed in claim 1, wherein an inner lining is arranged on the lid and the sterilizing device is arranged on the inner lining, with a first sealing ring being arranged at one end of the inner lining close to the box body.

9. The sterilization box as claimed in claim 8, wherein the inner lining comprises a hinge formed on an edge of one side thereof and hinged to a side of the box body via a rotating shaft; the hinge comprising a second spring formed therein and passing through the rotating shaft, and a pair of bending portions formed by two ends of the second spring and extending reversely and in parallel, one bending portion abutted against the inner lining and the other bending portion abutted against a top surface of the box body.

10. The sterilization box as claimed in claim 9, wherein a first electrode is arranged on the inner lining close to the hinge and electrically connected to the sterilizing device, and a second electrode is arranged on the top surface of the box body and corresponding to the first electrode; the box body further comprising a first battery, a first control circuit board and a charging electrode arranged on the bottom of the receiving room, all of the second electrode, the first control circuit board and the charging electrode electrically connected to the first battery, and the first electrode connected with the second electrode to drive the sterilizing device to work.

11. The sterilization box as claimed in claim 1, wherein the box body comprises a top surface, a bottom surface and a periphery sidewall connected to the top surface and the bottom surface, a U-shaped slope formed between the top surface and the periphery sidewall, and between the bottom surface and the periphery sidewall, respectively.

12. The sterilization box as claimed in claim 11, wherein the U-shaped slope comprises a pair of longitudinal slopes formed in parallel and opposite to each other, and a transverse slope connected with the pair of longitudinal slopes, an arc structure formed on a connection of the longitudinal slope and the transverse slope.

13. The sterilization box as claimed in claim 12, wherein the lid comprises an upper surface, a lower surface and a lateral sidewall connected with the upper and lower surfaces, an inverted U-shaped slope formed between the upper surface and the lateral sidewall, and between the lower surface and the lateral sidewall, respectively; and the inverted U-shaped slope is connected with the U-shaped slope when the lid is covered on the box body.

14. An electronic cigarette assembly comprising a sterilization box and an electronic cigarette received in the sterilization box, the sterilization box comprising:
a box body comprising a receiving room for receiving the electronic cigarette therein;
a lid comprising a sterilizing device arranged at one end thereof towards the receiving room and configured to sterilize the electronic cigarette;
a locking device configured to lock the lid and the box body and comprising an inserting structure arranged in the box body, a clamping structure embedded in the lid and used for inserting into and clamping with the inserting structure; the inserting structure comprising a pressing member, a first spring, and an inserting member received in the box body and extending out of the box body, with a wedge-shaped upper surface thereof contacting with the pressing member; and the pressing member buckled with the inserting member and then pushing the inserting member to move along a longitudinal direction of the box body so that the inserting member is separated from the clamping structure to quickly open the lid and the box body; the first spring abutted against the pressing member to push the pressing member back to an original position of the pressing member; and
the electronic cigarette comprising a suction member, an atomizing core member, a base member and a host connected successively from top to bottom;
the suction member abutted against the host via an outer wall thereof and comprising an e-liquid storage tank;
the base member buckled and fixed with the suction member for sealing the e-liquid storage tank; and
the electronic cigarette comprising a tapered slope extending longitudinally from a top surface of the electronic cigarette through a sidewall of the electronic cigarette to a bottom surface of the electronic cigarette along a longitudinal direction of the electronic cigarette.

15. The electronic cigarette assembly as claimed in claim 14, wherein the tapered slope is extended from a middle position of the host to both ends of the host along the longitudinal direction of the electronic cigarette, and then gradually folded and aggregated at a distal end of the electronic cigarette to form a tapered portion, one end of the tapered portion extending to the suction member and the other opposite end of the tapered portion extending to the bottom of the host.

16. The electronic cigarette assembly as claimed in claim 14, wherein the atomizing core member comprises a heating member comprising a silicone case embedded in the base member to separate the base member from the e-liquid storage tank, and a heating wire passing through the silicone case and then fixed with the silicone case.

17. The electronic cigarette assembly as claimed in claim 16, wherein the atomizing core member further comprises an e-liquid feeding base buckled with the base member and abutted against the silicone case, the e-liquid feeding base comprising a pair of longitudinal e-liquid feeding holes connected with the e-liquid storage tank;
a pair of gaskets arranged between the e-liquid feeding base and the silicone base, and respectively embedded on the silicone base, each of the pair of gaskets comprising a first semicircular groove, and the e-liquid feeding base comprising a second semicircular groove formed on the bottom thereof and corresponding to the first semicircular groove, each first semicircular groove and each second semicircular groove cooperatively enclosed to form an opening so that the heating wire is formed between the pair of openings.

18. The electronic cigarette assembly as claimed in claim 17, wherein an air passage is formed on the suction member along the longitudinal direction of the electronic cigarette to divide the e-liquid storage tank into two halves, the e-liquid feeding base comprising a second through-hole connected with the air passage; a second sealing ring arranged on the e-liquid feeding base, an outer wall of the second sealing ring abutted against an inner wall of the suction member, and an inner wall of the second sealing ring abutted against the e-liquid feeding base and the air passage, respectively.

19. The electronic cigarette assembly as claimed in claim 16, wherein the host comprises a case and a frame fixed in the case and receiving a second battery therein, an upper end surface of the frame abutted against a bottom end surface of the base member, a third electrode embedded in the bottom end surface of the base member and electrically connected with the atomizing core member, a fourth electrode embedded in the upper end surface of the frame and electrically connected with the second battery, the third electrode electrically connected with the fourth electrode so as to supply power for the heating member and the atomizing core member; and
a magnet surrounded around the fourth electrode.

20. The electronic cigarette assembly as claimed in claim 19, wherein the electronic cigarette further comprises a second control circuit board positioned below the second battery and electrically connected to the second battery, a microphone electrically connected to the second control circuit board, and a microphone silicone base sleeved around the microphone.
